(19) United States
(12) Patent Application Publication
(10) Pub. No.: US 2026/0026766 A1
(43) Pub. Date: Jan. 29, 2026

(54) ANALYSIS METHOD AND ELECTRONIC DEVICE FOR CORONARY ARTERY IMAGE

(71) Applicants: COMPAL ELECTRONICS, INC., Taipei City (TW); Chi Mei Medical Center, Tainan City (TW)

(72) Inventors: Chieh-Hung Chang, Taipei City (TW); Jen-Sheng Huang, Taipei City (TW); Yuan-Hsing Hsu, Taipei City (TW); Meng-Che Tsai, Taipei City (TW); Nien-Lun Chen, Taipei City (TW); Shih-Hsu Huang, Taipei City (TW); Kun-Sung Chen, Taipei City (TW); Wei-Ting Chang, Tainan City (TW); Kuo-Ting Tang, Tainan City (TW); Zhih-Cherng Chen, Tainan City (TW)

(73) Assignees: COMPAL ELECTRONICS, INC., Taipei City (TW); Chi Mei Medical Center, Tainan City (TW)

(21) Appl. No.: 19/260,622

(22) Filed: Jul. 7, 2025

Related U.S. Application Data
(60) Provisional application No. 63/674,277, filed on Jul. 23, 2024.

(30) Foreign Application Priority Data
Jun. 6, 2025 (TW) 114121204

(51) Int. Cl. A61B 6/00; A61B 6/50; G06T 7/00; G06T 7/11; G06V 10/26; G06V 10/764; G06V 20/70; G06V 40/10; G16H 30/40

(52) U.S. Cl. CPC A61B 6/5217; A61B 6/504; G06T 7/0012; G06T 7/11; G06V 10/26; G06V 40/15; G16H 30/40; G06T 2207/20081; G06T 2207/30048; G06T 2207/30104; G06V 10/764; G06V 20/70; G06V 2201/031

(57) ABSTRACT

An analysis method and an electronic device for a coronary artery image are provided. The method includes: performing segmentation on the coronary artery image based on a machine learning model to obtain a plurality of categories; setting one of the categories as a currently evaluated vessel, and determining whether a pixel quantity corresponding to the currently evaluated vessel is less than a first threshold to generate a result; and determining whether the coronary artery image has an occlusion phenomenon according to the result.

### CROSS-REFERENCE TO RELATED APPLICATION

This application claims the priority benefit of U.S. provisional application Ser. No. 63/674,277, filed on Jul. 23, 2024, and Taiwan application serial no. 114121204, filed on Jun. 6, 2025. The entirety of each of the above-mentioned patent application is hereby incorporated by reference herein and made a part of this specification.

### BACKGROUND

### Technical Field

The disclosure relates to an analysis method and electronic device for a coronary artery image using a machine learning model.

### Description of Related Art

Chronic total occlusion (CTO) of the coronary artery is a clinically highly challenging cardiovascular disease, characterized primarily by complete occlusion of the coronary artery with a duration typically exceeding three months. The presence of CTO lesions is highly correlated with various heart disease risks such as myocardial ischemia, angina symptoms, and decreased left ventricular function. If not diagnosed and processed in time, it may lead to decreased quality of life in patients or even increased risk of cardiovascular events.

Currently, coronary angiography (CAG) is commonly used as the main auxiliary tool for determining CTO in clinical practice. CAG may clearly show the occlusion condition of coronary artery through contrast agent imaging. However, such technology highly relies on experienced interventional cardiologists or radiology specialists to perform interpretation, and the diagnostic results are easily affected by operator experience and subjective judgment, which may lead to insufficient consistency and risk of misjudgment.

### SUMMARY

To solve the above problems, the disclosure provides an analysis method and an electronic device for a coronary artery image.

The disclosure provides an analysis method for a coronary artery image, performed by an electronic device. The analysis method includes: performing segmentation on the coronary artery image based on a machine learning model to obtain a plurality of categories; setting one of the categories as a currently evaluated vessel, and determining whether a pixel quantity corresponding to the currently evaluated vessel is less than a first threshold to generate a result; and determining whether the coronary artery image has an occlusion phenomenon according to the result.

In an embodiment of the disclosure, the step of determining whether the coronary artery image has the occlusion phenomenon according to the result includes: if the pixel quantity corresponding to the currently evaluated vessel is less than the first threshold, obtaining a distal vessel corresponding to the currently evaluated vessel from among the categories; determining whether a pixel quantity corresponding to the distal vessel is less than a second threshold; and if the pixel quantity corresponding to the distal vessel is less than the second threshold, determining that the coronary artery image has the occlusion phenomenon.

In an embodiment of the disclosure, the step of determining whether the coronary artery image has the occlusion phenomenon according to the result includes: if the pixel quantity corresponding to the distal vessel is greater than or equal to the second threshold, setting the distal vessel as the currently evaluated vessel, and repeating the step of determining whether the pixel quantity corresponding to the currently evaluated vessel is less than the first threshold.

In an embodiment of the disclosure, the step of determining whether the coronary artery image has the occlusion phenomenon according to the result further includes: if the pixel quantity corresponding to the currently evaluated vessel is greater than or equal to the first threshold, determining whether there is the distal vessel or a branch vessel corresponding to the currently evaluated vessel among the categories; if there is no distal vessel and branch vessel corresponding to the currently evaluated vessel among the categories, then determining that the coronary artery image does not have the occlusion phenomenon.

In an embodiment of the disclosure, the step of determining whether the coronary artery image has the occlusion phenomenon according to the result further includes: if there is the distal vessel or the branch vessel corresponding to the currently evaluated vessel among the categories, setting the distal vessel or the branch vessel as the currently evaluated vessel, and repeating the step of determining whether the pixel quantity corresponding to the currently evaluated vessel is less than the first threshold.

In an embodiment of the disclosure, the first threshold is the same as the second threshold.

In an embodiment of the disclosure, the analysis method further includes: calculating a total pixel quantity corresponding to all of the categories; and multiplying the total pixel quantity by a ratio to obtain the first threshold.

In an embodiment of the disclosure, the coronary artery image belongs to a right coronary artery image, a left anterior descending image, or a left circumflex image.

In an embodiment of the disclosure, the analysis method further includes: before performing segmentation on the coronary artery image, performing preprocessing on the coronary artery image, where the preprocessing includes blurring or contrast enhancement.

In an embodiment of the disclosure, the analysis method further includes: determining a location of the occlusion phenomenon according to a location of the currently evaluated vessel.

From another perspective, an embodiment of the disclosure provides an electronic device, including a memory and a processor. The memory stores a plurality of instructions. The processor is electrically connected to the memory for executing the instructions to complete the analysis method.

In order to make the above-mentioned features and advantages of the disclosure clearer and easier to understand, the following embodiments are given and described in details with accompanying drawings as follows.

### BRIEF DESCRIPTION OF THE DRAWINGS

FIG. 1 is a schematic diagram illustrating an electronic device according to an embodiment.

FIG. 2 is a flowchart illustrating an analysis method of a coronary artery image according to an embodiment.

FIG. 3 is an example of an LAD image and markers according to an embodiment.

FIG. 4 is an example of an LCX image and markers according to an embodiment.

FIG. 5 is an example of an RCA image and markers according to an embodiment.

FIG. 6 is a flowchart illustrating an analysis method of a coronary artery image according to another embodiment.

FIG. 7 is a flowchart illustrating a process of processing an LAD image according to an embodiment.

FIG. 8 is a flowchart illustrating a process of processing an LCX image according to an embodiment.

FIG. 9 is a flowchart illustrating a process of processing an RCA image according to an embodiment.

### DESCRIPTION OF THE EMBODIMENTS

Some embodiments of the disclosure accompanied with drawings are described in detail as follows. The reference numerals used in the following description are regarded as the same or similar elements when the same reference numerals appear in different drawings. These embodiments are only a part of the disclosure, and do not disclose all the possible implementation modes of the disclosure. To be more precise, the embodiments are only examples of the systems and methods in the scope of the claims of the disclosure.

Moreover, terms such as “first” and “second” used herein do not represent order or sequence, but are merely used for differentiating elements or operations having the same technical terms.

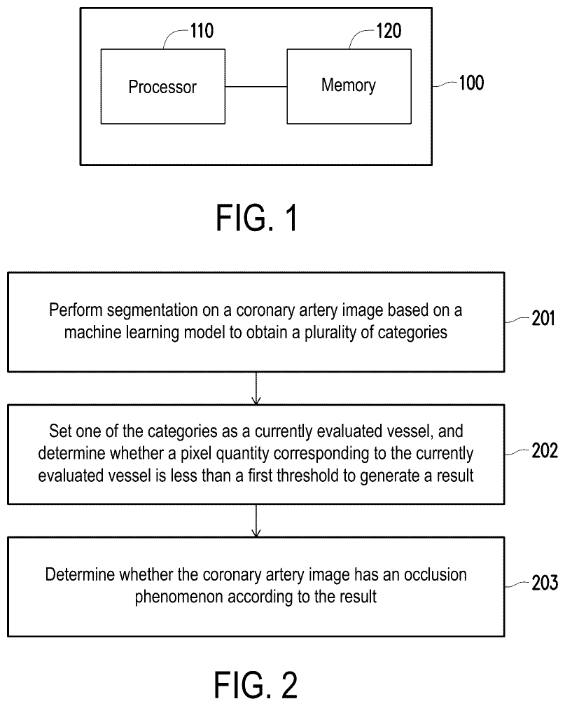

FIG. 1 is a schematic diagram illustrating an electronic device according to an embodiment. Referring to FIG. 1, an electronic device 100 may be a tablet computer, personal computer, laptop computer, server, cloud server, medical equipment, various electronic devices with computing capability, etc., and the disclosure is not limited thereto. The electronic device 100 includes a processor 110 and a memory 120, and the processor 110 is electrically connected to the memory 120. The processor 110 may be a central processing unit, image processing chip, deep-learning processing unit (DPU), neural network processing unit (NPU), tensor processing unit (TPU), application specific integrated circuit (ASIC), programmable logic device (PLD), etc. The memory 120 may be random access memory, read-only memory, flash memory, floppy disk, hard disk, optical disk, USB drive, magnetic tape, or a database accessible through the Internet, which stores a plurality of instructions, and the processor 110 executes the instructions to complete an analysis method of a coronary artery image.

FIG. 2 is a flowchart illustrating an analysis method of a coronary artery image according to an embodiment. Referring to FIG. 2, in step 201, segmentation is performed on a coronary artery image based on a machine learning model to obtain a plurality of categories. The coronary artery image is also referred to as coronary angiography (CAG). The machine learning is, for example, multi-layer neural network, convolutional neural network, support vector machine, etc. The architecture of the convolutional neural network may adopt LeNet, AlexNet, VGG, GoogLeNet, ResNet, DenseNet, U-Net, or YOLO (You Only Look Once), etc., and the disclosure is not limited thereto. In the training stage, professionals mark each vessel in the coronary artery image. According to the shooting angle, a coronary artery image may belong to a right coronary artery (RCA) image, a left anterior descending (LAD) image, or a left circumflex (LCX) image.

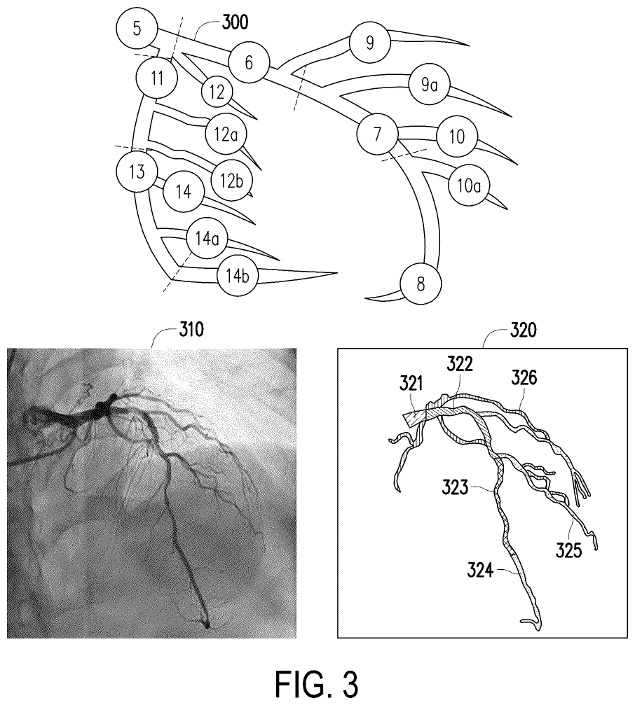

FIG. 3 is an example of an LAD image and markers according to an embodiment. Referring to FIG. 3, a classification schematic diagram 300 contains the numbers of each vessel, and according to the syntax score, all vessels may be numbered 5 to 14. Some vessels also have branches, and for example, some vessels are numbered 9a, 10a, 12a, 12b, 14a, 14b, etc. A coronary artery image 310 is the image before segmentation is performed. In the embodiment, the coronary artery image 310 is a grayscale image (only one channel), but in other embodiments it may also be a color image. There are 6 categories in total, and an image 320 already has corresponding markers (i.e., categories 321 to 326). The first category 321 contains number 5; the second category 322 contains number 6; the third category 323 contains number 7; the fourth category 324 contains number 8; the fifth category 325 contains numbers 9, 9a, 10, and 10a; and the sixth category 326 contains other numbers. In the training stage, the coronary artery image 310 serves as the input of the machine learning model, while the marked image 320 serves as the output of the machine learning model.

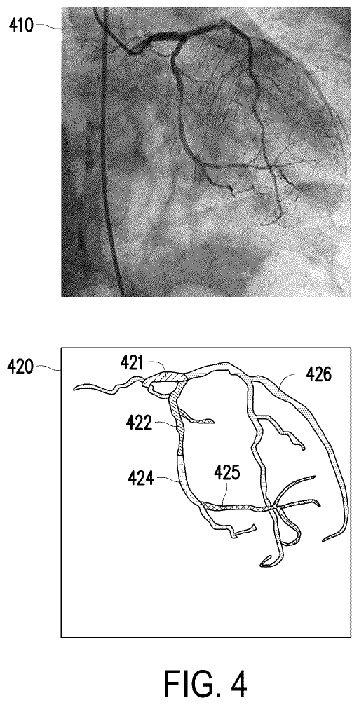

FIG. 4 is an example of an LCX image and markers according to an embodiment. Referring to FIG. 3 and FIG. 4, according to the classification schematic diagram 300, there are 6 categories in total in this example. The first category 421 contains number 5; the second category 422 contains number 11; the third category contains number 12 (since each person's vessel structure is different, this category does not exist in image 420); the fourth category 424 contains number 13; the fifth category 425 contains numbers 12a, 12b, 14, 14a, and 14b; and the sixth category 426 contains other numbers. In the training stage, the coronary artery image 410 serves as the input of the machine learning model, while the marked image 420 serves as the output of the machine learning model.

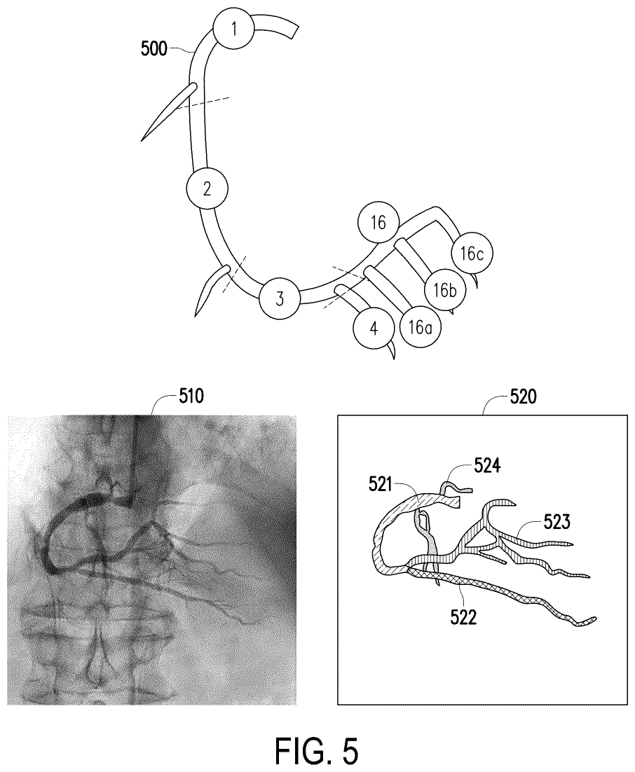

FIG. 5 is an example of an RCA image and markers according to an embodiment. A classification schematic diagram 500 shows the numbers of each vessel. There are 4 categories in total in this example. The first category 521 contains numbers 1, 2, and 3; the second category 522 contains number 4; the third category 523 contains numbers 16, 16a, 16b, and 16c; and the fourth category 524 contains other numbers. In the training stage, the coronary artery image 510 serves as the input of the machine learning model, while the marked image 520 serves as the output of the machine learning model.

In some embodiments, the RCA image, LAD image, and LCA are respectively processed by three different machine learning models, with each machine learning model focusing on a certain angle. In other embodiments, the same machine learning model may also be used to process images from all angles. In the above examples, the vessels in each view may be divided into 4 or 6 categories, but the disclosure does not limit the number of categories in each view.

In the above examples, each category corresponds to one or more pixels in the coronary artery image 310. However, in some examples during the inference stage, some vessels may not be classified into any category due to severe occlusion. In other words, some categories in the output of the machine learning model may not have corresponding pixels.

Here, the relationship between each category may be established from proximal to distal on the same vessel. For example, referring to the classification schematic diagram 300, in the LAD image, the vessel with number 5 is proximal, followed by the vessel with number 6, then the vessel with number 7, and the most distal is the vessel with number 8. Such proximal-distal relationship is relative. For example, relative to the vessel with number 5, the vessel with number 6 may be referred to as a distal vessel; relative to the vessel with number 6, the vessel with number 7 may be referred to as a distal vessel, and so on. The above relationship may be indicated as 5→6→7→8.

Similarly, in the LCX image, relationships may also be established according to proximity. The vessel with number 5 is proximal, followed by the vessel with number 11, then the vessel with number 13. Such relationship may be indicated as 5→11→13.

Referring to the classification schematic diagram 500, in the RCA image, the vessels with numbers 1, 2, and 3 are proximal, followed by two branches. The first branch contains the vessel with number 4, and the second branch contains the vessels with numbers 16, 16a, 16b, and 16c. Such relationship may be indicated as 1, 2, 3→4 (first branch) and 1, 2, 3→16, 16a, 16b, 16c (second branch).

In some embodiments, before inputting the coronary artery image to the machine learning model, some preprocessing may also be performed on the coronary artery image first. The preprocessing may include blurring, contrast enhancement, denoising, and so on. For example, blurring may include a low-pass filter, and contrast enhancement may include local histogram equalization, but the disclosure is not limited thereto.

Referring to FIG. 2, next step 202 is performed to set one of the categories as a currently evaluated vessel, and determine whether a pixel quantity corresponding to the currently evaluated vessel is less than a first threshold to generate a result. Each category is a segment of vessel. In the segmentation result, if a category has a plurality of pixels, it indicates that the corresponding vessel is relatively clear. If a certain category has fewer pixels, the corresponding vessel may have occlusion. If a certain category has no pixels, it indicates that the corresponding vessel has no pixels belonging to this category due to severe occlusion. Here, the pixel quantity of a category indicates the number of pixels belonging to the category, and the pixel quantity will be greater than or equal to 0. When the pixel quantity corresponding to a certain category is too small, it may represent that the segment of vessel has occlusion. Here, a first threshold is set to determine whether the pixel quantity corresponding to a certain category (i.e., the currently evaluated vessel) is less than the first threshold. The first threshold may be static or dynamic. For example, the total number of pixels corresponding to all of the categories may be calculated (referred to as total pixel quantity), and then the total pixel quantity is multiplied by a ratio (for example, 5%, 10%, or other values) to obtain the first threshold.

In step 203, it is determined whether the coronary artery image has an occlusion phenomenon according to the result generated in step 202. As described above, when the pixel quantity corresponding to a certain category is too small, an occlusion phenomenon may occur. In some embodiments, whether there is an occlusion phenomenon may be determined according to the determination result of one or a plurality of categories. For example, when the pixel quantity corresponding to the proximal vessel is less than the first threshold, it may be further determined whether the pixel quantity corresponding to the distal vessel is also too small.

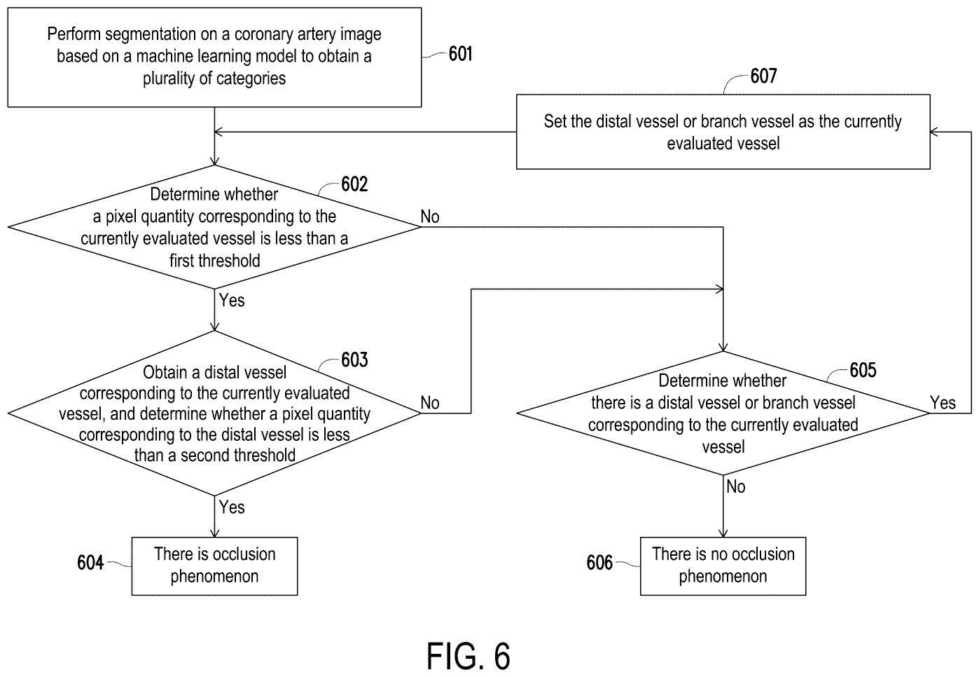

FIG. 6 is a flowchart illustrating an analysis method of a coronary artery image according to another embodiment. Referring to FIG. 6, step 601 is the same as step 201, and will not be repeated here. Next, a most proximal vessel is taken as the currently evaluated vessel, and then in step 602, it is determined whether the pixel quantity corresponding to the currently evaluated vessel is less than the first threshold.

If the determination result of step 602 is yes, in step 603, the distal vessel corresponding to the currently evaluated vessel is obtained, and it is determined whether the pixel quantity corresponding to the distal vessel is less than a second threshold. For example, in FIG. 3, if the pixel quantity corresponding to the category 321 is less than the first threshold, then the categories 322 to 324 are taken as distal vessels. In some embodiments, all distal vessels may be determined (all the way to the end of the vessel), but in other embodiments, only one distal vessel may be determined (for example, category 322). The second threshold may be the same as or different from the first threshold. In some embodiments, the second threshold may be less than the first threshold. If the result of step 603 is yes, this indicates that the pixel quantities corresponding to both the proximal vessel and the distal vessel are small. Therefore, in step 604, it is determined that the coronary artery image has an occlusion phenomenon.

If the result of step 602 is no, or the result of step 603 is no, the process enters step 605, so as to determine whether there is the distal vessel or a branch vessel corresponding to the currently evaluated vessel. If step 603 has already been executed, it indicates that there is already a distal vessel. If the result of step 605 is no, it indicates that all vessels from proximal to distal have been analyzed. Therefore, in step 606, it is determined that the coronary artery image does not have an occlusion phenomenon.

If the result of step 605 is yes, then the distal vessel or the branch vessel is set as the currently evaluated vessel. Next, step 602 is repeated. In other words, here the analysis starts from the proximal vessel, updating the currently evaluated vessel in a loop manner until processing to the end of the vessel. According to this approach, it may objectively determine whether the coronary artery has an occlusion phenomenon. Multiple examples will be given below for further illustration.

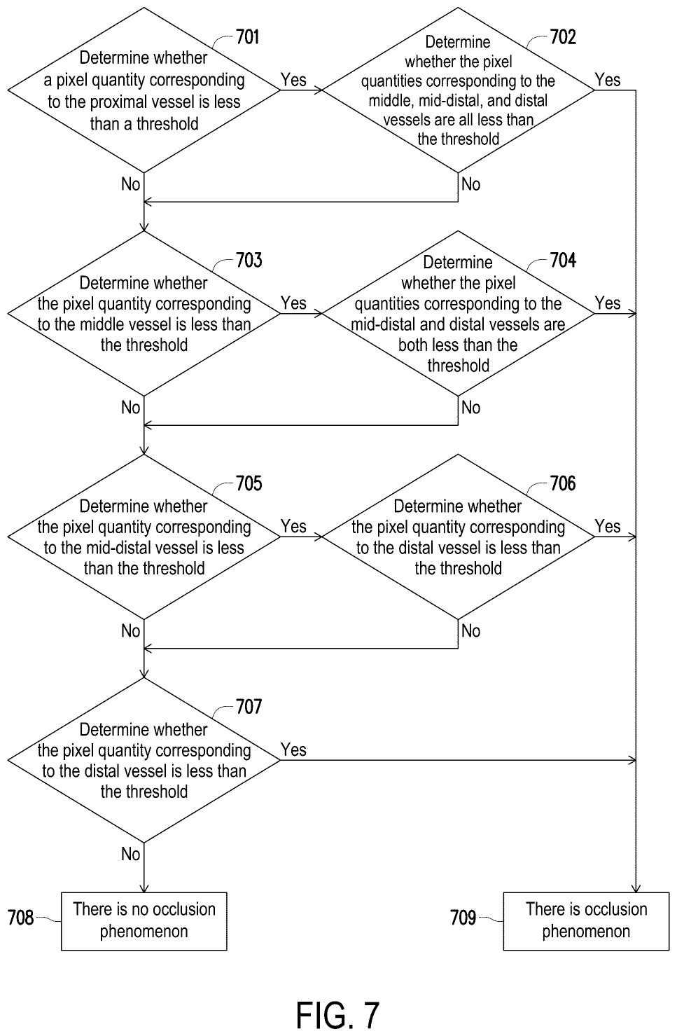

FIG. 7 is a flowchart illustrating a process of processing an LAD image according to an embodiment. Referring to FIG. 3 and FIG. 7, as described above, the proximal-distal relationship of vessels in the LAD image is indicated as 5→6→7→8, with a total of 4 categories. For convenience, these 4 categories are respectively referred to as proximal vessel, middle vessel, mid-distal vessel, and distal vessel. In addition, in this example, the first threshold is the same as the second threshold. Here the loop of FIG. 6 is unfolded into steps 701 to 709. In step 701, it is determined whether the pixel quantity corresponding to the proximal vessel is less than the threshold. If yes, then the process proceeds to step 702; otherwise, the process proceeds to step 703. In step 702, it is determined whether the pixel quantities corresponding to the middle, mid-distal, and distal vessels are all less than the threshold. If yes, then the process proceeds to step 709; otherwise, the process proceeds to step 703. In step 703, it is determined whether the pixel quantity corresponding to the middle vessel is less than the threshold. If yes, then the process proceeds to step 704; otherwise, the process proceeds to step 705. In step 704, it is determined whether the pixel quantities corresponding to the mid-distal and distal vessels are both less than the threshold. If yes, then the process proceeds to step 709; otherwise, the process proceeds to step 705. In step 705, it is determined whether the pixel quantity corresponding to the mid-distal vessel is less than the threshold. If yes, then the process proceeds to step 706; otherwise, the process proceeds to step 707. In step 706, it is determined whether the pixel quantity corresponding to the distal vessel is less than the threshold. If yes, then the process proceeds to step 709; otherwise, the process proceeds to step 707. In step 707, it is determined whether the pixel quantity corresponding to the distal vessel is less than the threshold. If yes, then the process proceeds to step 709; otherwise, the process proceeds to step 708. In step 708, it is determined that there is no occlusion phenomenon. In step 709 it is determined that there is an occlusion phenomenon.

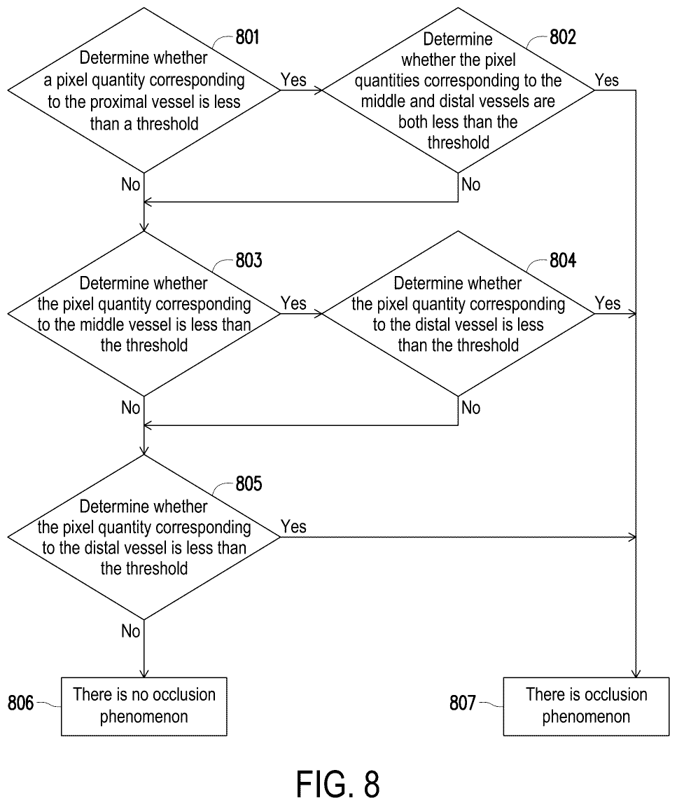

FIG. 8 is a flowchart illustrating a process of processing an LCX image according to an embodiment. Referring to FIG. 4 and FIG. 8, as described above, the proximal-distal relationship of vessels in the LCX image is indicated as 5→11→13, with a total of 3 categories. For convenience, these 3 categories are respectively referred to as proximal vessel, middle vessel, and distal vessel. Similarly, here the loop of FIG. 6 is unfolded into steps 801 to 807. In step 801, it is determined whether the pixel quantity corresponding to the proximal vessel is less than the threshold. If yes, then the process proceeds to step 802; otherwise, the process proceeds to step 803. In step 802, it is determined whether the pixel quantities corresponding to the middle and distal vessels are both less than the threshold. If yes, then the process proceeds to step 807; otherwise, the process proceeds to step 803. In step 803, it is determined whether the pixel quantity corresponding to the middle vessel is less than the threshold. If yes, then the process proceeds to step 804; otherwise, the process proceeds to step 805. In step 804, it is determined whether the pixel quantity corresponding to the distal vessel is less than the threshold. If yes, then the process proceeds to step 807; otherwise, the process proceeds to step 805. In step 805, it is determined whether the pixel quantity corresponding to the distal vessel is less than the threshold. If yes, then the process proceeds to step 807; otherwise, the process proceeds to step 806. In step 806 it is determined that there is no occlusion phenomenon. In step 807 it is determined that there is an occlusion phenomenon.

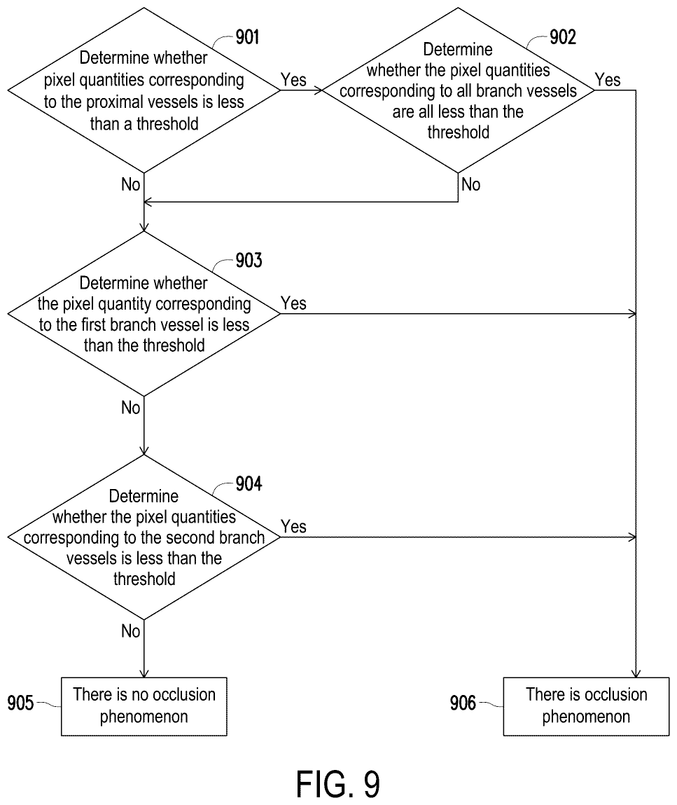

FIG. 9 is a flowchart illustrating a process of processing an RCA image according to an embodiment. As described above, there are two branches in the RCA image. The first branch is 1, 2, 3→4, and the second branch is 1, 2, 3→16, 16a, 16b, 16c. Here the vessels with numbers 1, 2, 3 are referred to as proximal vessels, the vessel with number 4 is referred to as the first branch vessel, and the vessels with numbers 16, 16a, 16b, 16c are referred to as the second branch vessels. Similarly, here the loop of FIG. 6 is unfolded into steps 901 to 906. In step 901, it is determined whether the pixel quantities corresponding to the proximal vessels is less than the threshold. If yes, then the process proceeds to step 902; otherwise, the process proceeds to step 903. In step 902, it is determined whether the pixel quantities corresponding to all branch vessels are all less than the threshold. If yes, then the process proceeds to step 906; otherwise, the process proceeds to step 903. In step 903, it is determined whether the pixel quantity corresponding to the first branch vessel is less than the threshold. If yes, then the process proceeds to step 906; otherwise, the process proceeds to step 904. In step 904, it is determined whether the pixel quantities corresponding to the second branch vessels is less than the threshold. If yes, then the process proceeds to step 906; otherwise, the process proceeds to step 905. In step 905, it is determined that there is no occlusion phenomenon. In step 906, it is determined that there is an occlusion phenomenon.

In some embodiments, the location where the occlusion phenomenon occurs may also be determined according to the location of the currently evaluated vessel. For example, in FIG. 7, if step 709 is entered from step 702, it indicates that the occlusion phenomenon occurs in the proximal vessel (the currently evaluated vessel belongs to the proximal vessel). If step 709 is entered from step 704, it indicates that the occlusion phenomenon occurs in the middle vessel. If step 709 is entered from step 706, it indicates that the occlusion phenomenon occurs in the mid-distal vessel. If step 709 is entered from step 707, it indicates that the occlusion phenomenon occurs in the distal vessel.

In FIG. 8, if step 807 is entered from step 802, it indicates that the occlusion phenomenon occurs in the proximal vessel. If step 807 is entered from step 804, it indicates that the occlusion phenomenon occurs in the middle vessel. If step 807 is entered from step 805, it indicates that the occlusion phenomenon occurs in the distal vessel.

In FIG. 9, if step 906 is entered from step 902, it indicates that the occlusion phenomenon occurs in the proximal vessel. If step 906 is entered from step 903, it indicates that the occlusion phenomenon occurs in the first branch vessel. If step 906 is entered from step 904, it indicates that the occlusion phenomenon occurs in the second branch vessels.

According to the technical means disclosed above, objective algorithms may be used to determine whether a coronary artery image have an occlusion phenomenon, and the location of the occlusion phenomenon may also be accurately determined. These methods may assist medical personnel, for example, by alerting them to occlusion locations that require reexamination by medical personnel.

Although the disclosure has been described with reference to the embodiments above, the embodiments are not intended to limit the disclosure. Any person skilled in the art can make some changes and modifications without departing from the spirit and scope of the disclosure. Therefore, the scope of the disclosure will be defined in the appended claims.

What is claimed is:

1. An analysis method for a coronary artery image, performed by an electronic device, the analysis method comprising:
performing segmentation on the coronary artery image based on a machine learning model to obtain a plurality of categories;
setting one of the categories as a currently evaluated vessel, and determining whether a pixel quantity corresponding to the currently evaluated vessel is less than a first threshold to generate a result; and
determining whether the coronary artery image has an occlusion phenomenon according to the result.

2. The analysis method according to claim 1, wherein the step of determining whether the coronary artery image has the occlusion phenomenon according to the result comprises:
if the pixel quantity corresponding to the currently evaluated vessel is less than the first threshold, obtaining a distal vessel corresponding to the currently evaluated vessel from among the categories;
determining whether a pixel quantity corresponding to the distal vessel is less than a second threshold; and
if the pixel quantity corresponding to the distal vessel is less than the second threshold, determining that the coronary artery image has the occlusion phenomenon.

3. The analysis method according to claim 2, wherein the step of determining whether the coronary artery image has the occlusion phenomenon according to the result comprises:
if the pixel quantity corresponding to the distal vessel is greater than or equal to the second threshold, setting the distal vessel as the currently evaluated vessel, and repeating the step of determining whether the pixel quantity corresponding to the currently evaluated vessel is less than the first threshold.

4. The analysis method according to claim 2, wherein the step of determining whether the coronary artery image has the occlusion phenomenon according to the result further comprises:
if the pixel quantity corresponding to the currently evaluated vessel is greater than or equal to the first threshold, determining whether there is the distal vessel or a branch vessel corresponding to the currently evaluated vessel among the categories;
if there is no distal vessel and branch vessel corresponding to the currently evaluated vessel among the categories, then determining that the coronary artery image does not have the occlusion phenomenon.

5. The analysis method according to claim 4, wherein the step of determining whether the coronary artery image has the occlusion phenomenon according to the result further comprises:
if there is the distal vessel or the branch vessel corresponding to the currently evaluated vessel among the categories, setting the distal vessel or the branch vessel as the currently evaluated vessel, and repeating the step of determining whether the pixel quantity corresponding to the currently evaluated vessel is less than the first threshold.

6. The analysis method according to claim 5, wherein the first threshold is the same as the second threshold.

7. The analysis method according to claim 6, further comprising:
calculating a total pixel quantity corresponding to all of the categories; and
multiplying the total pixel quantity by a ratio to obtain the first threshold.

8. The analysis method according to claim 1, wherein the coronary artery image belongs to a right coronary artery image, a left anterior descending image, or a left circumflex image.

9. The analysis method according to claim 1, further comprising:
performing a preprocessing on the coronary artery image before performing segmentation on the coronary artery image, wherein the preprocessing comprises a blurring or a contrast enhancement.

10. The analysis method according to claim 1, further comprising:
determining a location of the occlusion phenomenon according to a location of the currently evaluated vessel.

11. An electronic device, comprising:
a memory, storing a plurality of instructions; and
a processor, electrically connected to the memory for executing the instructions to complete a plurality of steps:
performing segmentation on the coronary artery image based on a machine learning model to obtain a plurality of categories;
setting one of the categories as a currently evaluated vessel, and determining whether a pixel quantity corresponding to the currently evaluated vessel is less than a first threshold to generate a result; and
determining whether the coronary artery image has an occlusion phenomenon according to the result.

12. The electronic device according to claim 11, wherein the step of determining whether the coronary artery image has the occlusion phenomenon according to the result comprises:
if the pixel quantity corresponding to the currently evaluated vessel is less than the first threshold, obtaining a distal vessel corresponding to the currently evaluated vessel from among the categories;
determining whether a pixel quantity corresponding to the distal vessel is less than a second threshold; and
if the pixel quantity corresponding to the distal vessel is less than the second threshold, determining that the coronary artery image has the occlusion phenomenon.

13. The electronic device according to claim 12, wherein the step of determining whether the coronary artery image has the occlusion phenomenon according to the result comprises:
if the pixel quantity corresponding to the distal vessel is greater than or equal to the second threshold, setting the distal vessel as the currently evaluated vessel, and repeating the step of determining whether the pixel quantity corresponding to the currently evaluated vessel is less than the first threshold.

14. The electronic device according to claim 12, wherein the step of determining whether the coronary artery image has the occlusion phenomenon according to the result further comprises:
if the pixel quantity corresponding to the currently evaluated vessel is greater than or equal to the first threshold, determining whether there is the distal vessel or a branch vessel corresponding to the currently evaluated vessel among the categories;
if there is no distal vessel and branch vessel corresponding to the currently evaluated vessel among the categories, then determining that the coronary artery image does not have the occlusion phenomenon.

15. The electronic device according to claim 14, wherein the step of determining whether the coronary artery image has the occlusion phenomenon according to the result further comprises:
if there is the distal vessel or the branch vessel corresponding to the currently evaluated vessel among the categories, setting the distal vessel or the branch vessel as the currently evaluated vessel, and repeating the step of determining whether the pixel quantity corresponding to the currently evaluated vessel is less than the first threshold.

16. The electronic device according to claim 15, wherein the first threshold is the same as the second threshold.

17. The electronic device according to claim 16, wherein the steps further comprise:
calculating a total pixel quantity corresponding to all of the categories; and
multiplying the total pixel quantity by a ratio to obtain the first threshold.

18. The electronic device according to claim 11, wherein the coronary artery image belongs to a right coronary artery image, a left anterior descending image, or a left circumflex image.

19. The electronic device according to claim 11, wherein the steps further comprise:
performing a preprocessing on the coronary artery image before performing segmentation on the coronary artery image, wherein the preprocessing comprises a blurring or a contrast enhancement.

20. The electronic device according to claim 11, wherein the steps further comprise:
determining a location of the occlusion phenomenon according to a location of the currently evaluated vessel.
